(19) United States
(12) Patent Application Publication
(10) Pub. No.: US 2021/0017588 A1
(43) Pub. Date: Jan. 21, 2021

(54) AMPLIFICATION OF NUCLEIC ACIDS

(71) Applicant: RevoluGen Limited, Hadfield, Derbyshire (GB)

(72) Inventors: Georgios PATSOS, Hadfield, Derbyshire (GB); Stephen John MINTER, Hadfield, Derbyshire (GB)

(21) Appl. No.: 16/341,768

(22) PCT Filed: Oct. 16, 2017
(86) PCT No.: PCT/GB2017/053128

§ 371 Date: Apr. 12, 2019

(30) Foreign Application Priority Data
Oct. 14, 2016 (GB) 1617491.4

(51) Int. Cl. C12Q 1/6844; C12Q 1/6816

(52) U.S. Cl. CPC C12Q 1/6844; C12Q 2521/319; C12Q 1/6816

(57) ABSTRACT

There is provided a method of amplifying a nucleic acid sequence. The method comprises providing an amplification mixture comprising an exonuclease capable of digestion of a strand of a double stranded nucleic acid molecule from the 5′-end towards the 3′-end, a strand displacing polymerase, a double stranded nucleic acid molecule comprising first and second nucleic acid strands, a first nucleic acid primer, and nucleotides as appropriate to provide for amplification of the first nucleic acid sequence to be amplified. The method further comprises effecting the amplification reaction under conditions permitting digestion, exonuclease digestion and strand displacement polymerisation thereby producing a product mixture comprising an amplified amount of said first nucleic acid sequence. There is also provided a method of determining the presence or quantity of target nucleic acid sequence in a biological sample using a double stranded probe having a fluorophore on one strand and its quencher on the other and using denaturation and re-hybridisation to detect the target.

The present invention relates to the amplification of nucleic acids and has particular (but not necessarily exclusive) application to the production of amplified amounts of a particular target sequence for detection for the purposes of medical diagnosis procedures.

Many medical conditions are characterised by the presence (in the patient's body) of a nucleic acid having a particular nucleotide sequence. The nucleic acid sequence may, for example, be one present in a pathogenic bacteria, virus or other microorganism which has “invaded” the patient's body and which is responsible for an illness in the patient. In many such instances, the presence of the microorganism in the patient's body may be diagnosed by analysing a sample such as tissue, blood, urine, sputum etc from a patient for the presence (in the sample) of a nucleic acid sequence that characterises the microorganism. However, in many cases, the amount of the characterising nucleic acid sequence in the sample is very low and below detectable limits. As such, amplification procedures are employed to enhance the amount of the characteristic sequence (or a characteristic variant thereof, e.g. a DNA sequence derived from a characterising rRNA sequence) for the purposes of detection.

According to a first aspect of the present invention there is provided a method of amplifying a nucleic acid sequence comprising:
(a) providing an amplification mixture which comprises:
   (i) an exonuclease capable of effecting digestion of a strand of a double stranded nucleic acid molecule with the digestion being from the 5′-end of the strand towards the 3′-end,
   (ii) a strand displacing polymerase,
   (iii) a double stranded nucleic acid molecule comprising first and second nucleic acid strands hybridised to each other, said first strand incorporating a first nucleic acid sequence to be amplified and having a first 5′-end region which remote from its 5′-end has a nucleotide sequence resistant to digestion (under the conditions of the method) by the exonuclease defined as (i),
   (iv) a first nucleic acid primer having the same nucleotide sequence as said first end region of the first nucleic acid, and incorporating the same digestion resistant region, and
   (v) nucleotides as appropriate to provide for amplification of the first nucleic acid sequence to be amplified;
      and
(b) effecting the amplification reaction under conditions permitting digestion, exonuclease digestion and strand displacement polymerisation thereby producing a product mixture comprising an amplified amount of said first nucleic acid sequence.

The amplification method of the first aspect of the present invention is based on a combination of a number of features. In particular, the method utilises an inter-related combination of (a) a first nucleic acid primer having a digestion resistant region remote from its 5′-end, and (b) a double stranded nucleic acid comprising first and second nucleic acid strands hybridised to each other, the first strand incorporating the nucleic acid sequence to be amplified. The combination is such that the first nucleic acid strand has, extending from its 5′-end, a 5′-end region with the same nucleotide sequence as the first primer including the digestion resistant sequence.

In the amplification method, the exonuclease digests the 5′-end region of the first strand to, but not through, the digestion resistant region in the 5′-end region of the strand. As a result, the 3′-end of the second strand is exposed and provides a site for hybridisation of the first primer which, in hybridising to the 3′-end of the second strand, displaces the undigested portion of the 5′-end region of the first strand. The first primer is then extended by the action of the strand displacing polymerase to produce a copy of the first strand. The 5′-end region of the newly synthesised first strand can then be digested (by the exonuclease) and the described process in effect repeats itself.

The amplification method of the invention is preferably one conducted under isothermal conditions, e.g. at a temperature of 45° C. to 55° C. As such, the strand displacing polymerase is one capable of copying a template strand under isothermal conditions. Similarly, the exonuclease is one capable of effecting digestion under the preferred isothermal conditions. Preferred strand displacement polymerases for use in the invention are those that lack 3′ exonuclease activity. The strand displacing polymerase may be one of the Bst series, although there are other possibilities as discussed below. The exonuclease is preferably one that recognises a blunt end of a double stranded nucleic acid molecule. It is particularly preferred that the exonuclease is A-exonuclease which progressively degrades one strand of double stranded DNA in the 5′ and 3′ direction in the following order of preference for the configuration of the ends of the double stranded structure namely 5′-recessed>blunt<<5′-overhang with a 10× preference for phosphorylated rather than hydroxylated ends.

In one embodiment of the invention, the second strand of the double stranded nucleic acid molecule may be “normal” in that it does not have a digestion resistant region. A process in accordance with this embodiment is described below in more detail with reference to FIG. 1. In this embodiment of the invention, it is preferred that the 5′-end of the first strand and the 3′-end of the second strand together provide a blunt end for the double stranded nucleic acid molecule. Furthermore, it is preferred that the 5′-end of the first strand has a 5′-phosphate group and the exonuclease is one which, in the amplification mixture, preferentially digests a strand of a double stranded nucleic acid molecule that has a phosphate (PO₄) group at its 5′-end with the digestion being from that end of the strand towards the 3′-end thereof to liberate the 3′-end of the second strand for hybridisation of the primer thereto. In such an embodiment, it is preferred that the exonuclease is λ-exonuclease.

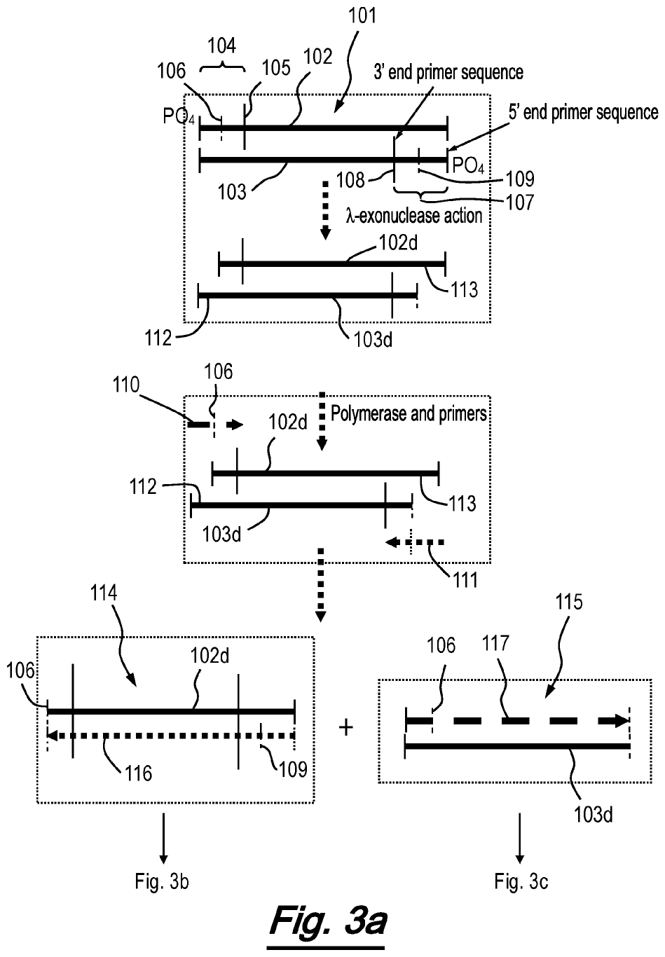

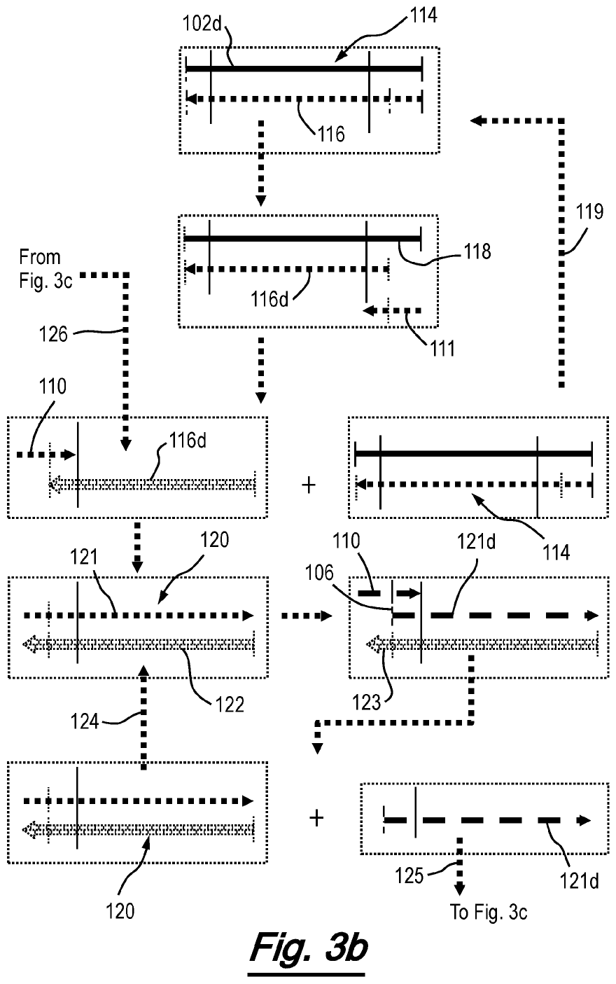

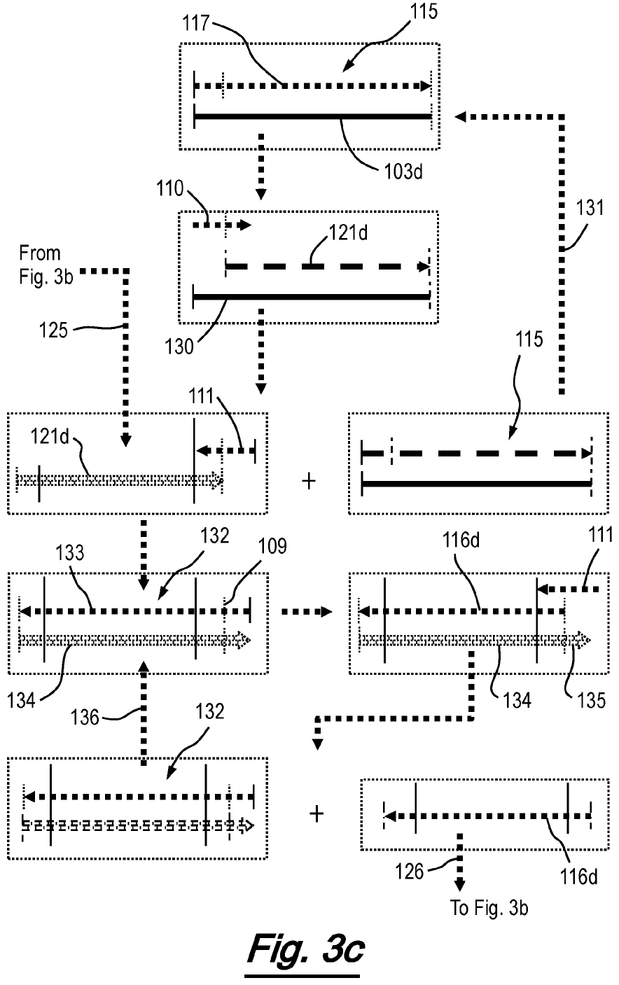

In a further embodiment of the invention, the method utilises a second nucleic acid primer having (like the first primer) a digestion resistant region remote from its 5′-end. Furthermore, in this embodiment the second strand has a 5′-end region extending from its 5′-end with the same nucleotide sequence as the second primer (including the digestion resistant sequence). This embodiment is described below in more detail with reference to FIG. 3 of the drawings. In this embodiment, it is preferred that the double stranded nucleic acid molecule has blunt ends. Preferably also the 5′-ends of each of the first and second strands have a 5′-phosphate group and the exonuclease is one which, in the amplification mixture, preferentially digests a strand of a double stranded nucleic acid molecule that has a phosphate (PO₄) group at is 5′-end with the digestion being from that end of the strand towards the 3′-end thereof. Preferably the exonuclease is λ-exonuclease.

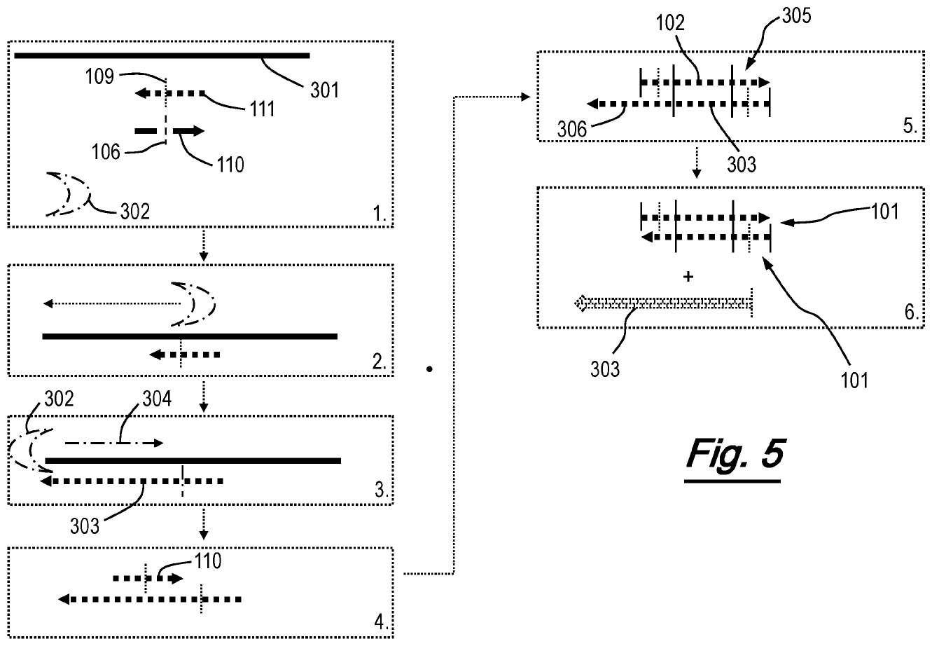

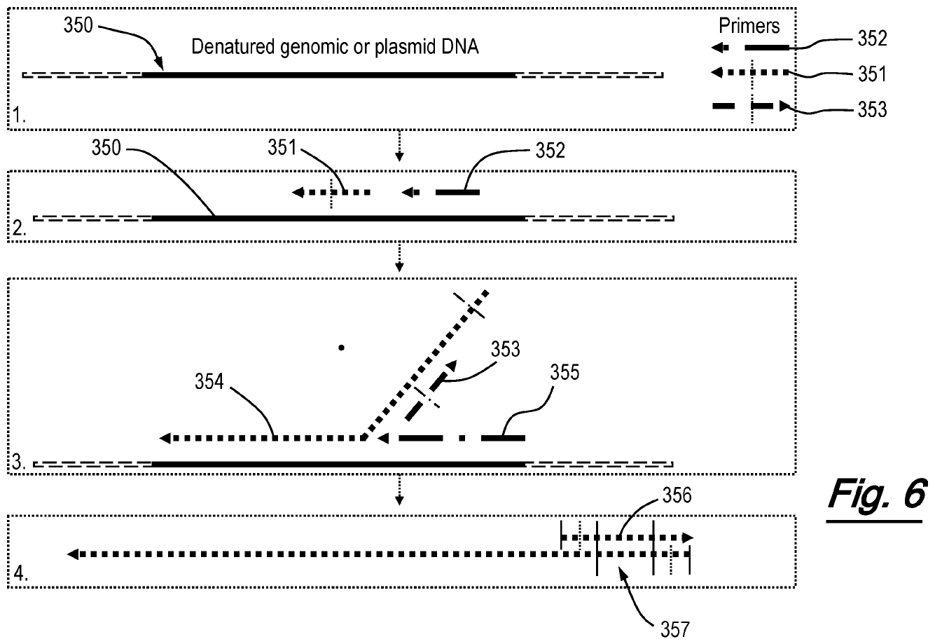

For all embodiments of the invention, the double stranded DNA molecule containing the sequence(s) to be amplified may be synthesised from a naturally occurring nucleic acid strand containing a sequence of interest. The naturally occurring strand may, for example, be one present in a bacteria or virus. The naturally occurring strand may, for example, be an rRNA strand. A procedure for obtaining a double stranded nucleic acid construct for use in the method of the invention from an rRNA strand is described below with reference to FIG. 5 of the drawings. Alternatively, the naturally occurring strand may be a DNA strand. In this case, the double stranded nucleic acid molecule to be amplified in accordance with the method of the invention may be derived from denatured genomic or plasmid DNA (see for example description below in relation to FIG. 6).

For all embodiments of the invention, it is preferred that the first primer (and also the second primer, if utilised) comprises 20 to 30 nucleotides. Correspondingly, the 5′-end region of the first strand (and, if utilised, the 5′-end region of the second strand) has a length of 20 to 30 nucleotides.

The digestion resistant region of the first primer (and second primer, if utilised) is preferably provided approximately mid-way along the length of the primer. Thus, in the case that the primer comprises 20 nucleotides, the digestion resistant region preferably starts about 8-10 nucleotides from 5′-end of the primer. In the case where the primer comprises 30 nucleotides the digestion resistant region preferably starts about 13 to 15 nucleotides from the 5′ end.

The digestion resistant regions may be provided by at least one nucleotide that is resistant to digestion by the exonuclease. Preferably the digestion resistant region comprises a consecutive sequence of a plurality (e.g. 3 to 6) of the modified nucleotides. The modified nucleotides may for example be phosphorothioate nucleotides (i.e. nucleotides in which a non-bridging oxygen atom is replaced by sulphur).

As indicated, it is preferred that the digestion resistant region (e.g. comprising a consecutive sequence of 3 to 6 modified nucleotides) is present approximately mid-way along the 1′ primer (and also 2′ if utilised). In such cases, it is particularly preferred that the strand displacement polymerase is one that lacks 3′ exonuclease activity. We do not however preclude the possibility that the digestion resistant region extends to the 3′ end or the primer in which case the use of a strand-displacing polymerase with 3′ exonuclease activity (e.g. phi29) may be employed.

Amplified sequences produced in accordance with the method of the invention may, for example, have a length of 50 to 150 bases per strand.

The method of the invention may further comprise the step of detecting the amplified sequence. In a preferred embodiment of the invention, detection is effected by the steps of:
(i) providing in the product mixture a nucleic acid reporter combination which comprises (a) a reporter strand having a fluorescent reporter moiety bound thereto, the reporter strand being capable of hybridising to the amplified nucleic acid sequence to be detected, and (b) a quencher strand capable of hybridising to the reporter strand and having a quencher moiety which quenches the fluorescence of the fluorescent reporter moiety,
(ii) subjecting the product mixture to denaturation and then rehybridisation conditions, and
(iii) detecting for the presence of the fluorescent reporter moiety.

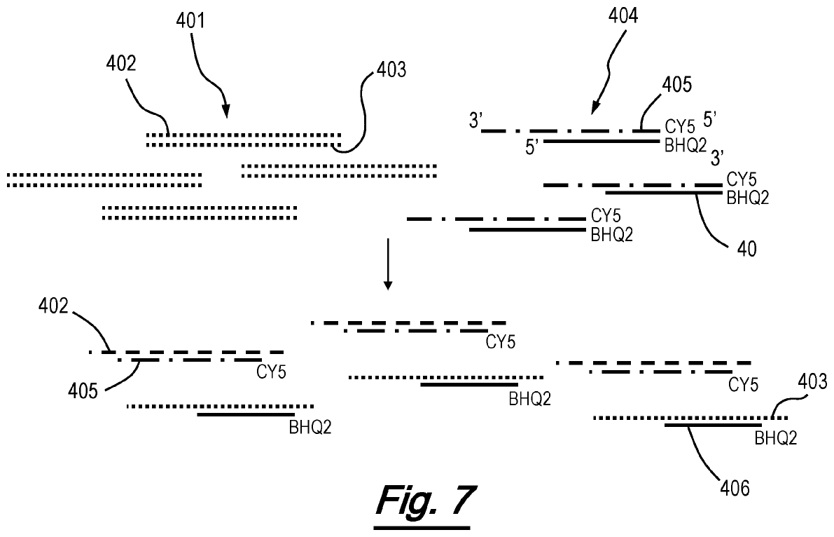

In step 2(ii) of this detection method, the product mixture is subjected successively to denaturation and then re-hybridisation conditions. Under the denaturation conditions, the reporter strand and quencher strand are separate strands within the product mixture. Under the re-hybridisation conditions, the reporter strand is able to hybridise to the amplified nucleic acid sequence, rather than to the quencher strand. Thus fluorescence after the re-hybridisation step confirms the presence of the amplified sequence. Generally, the quencher strand will be present in a molar excess as compared to the reporter strand. The molar ratio of quencher strand to reporter strand may, for example (1.3-1.5); 1. Embodiments of this detection method are described more fully below in conjunction with FIG. 7.

The method of the invention is applicable particularly, but by no means exclusively, to confirming the presence of a particular target nucleic acid sequence in a biological sample, for example tissue, blood, urine, sputum etc. The target nucleic acid may, for example, be one present in a pathogenic bacteria which is present in the tissue sample and which is responsible for illness of the patient. As outlined above, and as described in more detail below rRNA extracted from the sample may be used to prepare cDNA comprising first and second nucleic acid strands, hybridised to each other, said first strand incorporating a first nucleic acid sequence confirmatory of the presence of target nucleic acid sequence in the biological sample and having a first 5′-end region which remote from its 5′-end has a nucleotide sequence resistant to digestion. The cDNA may then be amplified using the procedures described more fully above.

This leads to a second aspect of the invention, according to which there is provided a method of determining the presence or otherwise of target nucleic acid sequence in a biological sample, the method comprising the steps of:
(a) processing the biological sample to produce therefrom a derivative sample and under conditions such that, if the target nucleic acid is present in the biological sample, there is generated in the derivative sample a double stranded nucleic acid molecule comprising first and second nucleic acid strands, hybridised to each other, said first strand incorporating a first nucleic acid sequence confirmatory of the presence of target nucleic acid sequence in the biological sample and having a first 5′-end region which remote from its 5′-end has a nucleotide sequence resistant to digestion;
(b) preparing an amplification composition which comprises:
   (i) an exonuclease capable of effecting digestion of a strand of a double stranded nucleic acid molecule with the digestion being from the 5′-end of the strand towards the 3′-end,
   (ii) a strand displacing polymerase,
   (iii) the derivative sample
   (iv) a first nucleic acid primer having the same nucleotide sequence as said first end region of the first nucleic acid if present in the derivative sample, and incorporating the same digestion resistant region, and
   (v) nucleotides as appropriate to provide for amplification of the first nucleic acid sequence to be amplified; and
(c) analysing for the presence of the first nucleic acid in the product mixture.

All features of the first aspect of the invention as described above are applicable mutatis mutandis to the method of the second aspect of the invention.

The derivative sample may, for example, comprise cDNA prepared from rRNA extracted from the biological sample.

The invention will be further described, by way of example only, with reference to the accompanying drawings, in which:

FIG. 1 schematically illustrates one embodiment of amplification method in accordance with the invention to illustrate the basic concept thereof;

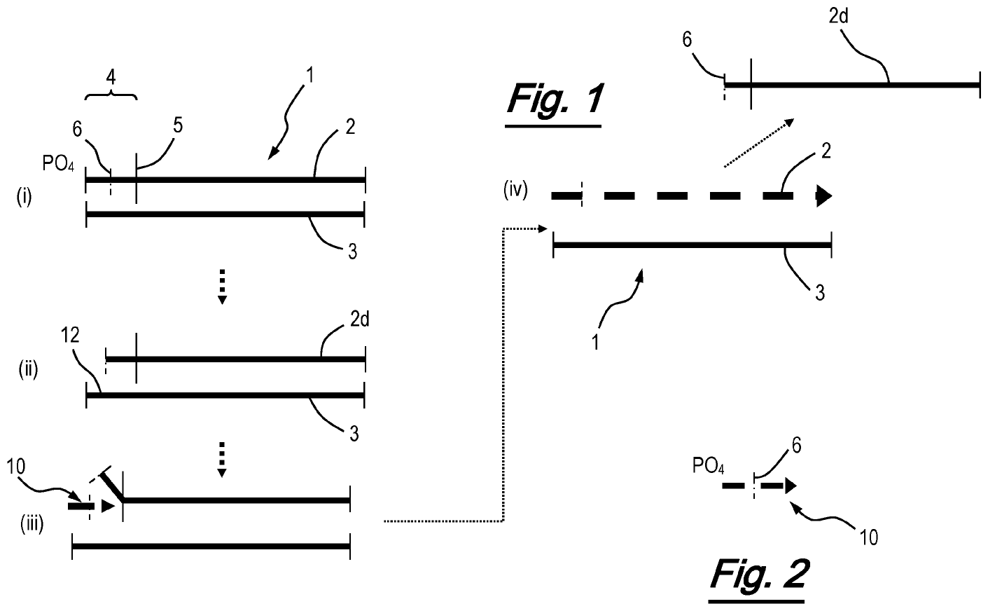

FIG. 2 schematically illustrates a primer for use in the method of FIG. 1;

FIGS. 3a-c illustrate a further embodiment of amplification method in accordance with the invention;

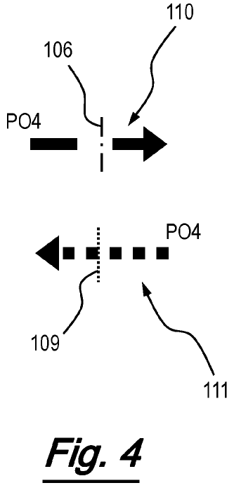

FIG. 4 schematically illustrates forward and reverse primers for use in the method illustrated in FIG. 3;

FIG. 5 illustrates production, from rRNA of a double stranded nucleic acid molecule for amplification in accordance with the procedure depicted in FIG. 3;

FIG. 6 depicts production, from genomic or plasmid DNA, of a double stranded DNA molecule for amplification in accordance with the procedure depicted in FIG. 3;

FIG. 7 illustrates an embodiment of procedure for detecting a nucleic acid molecule;

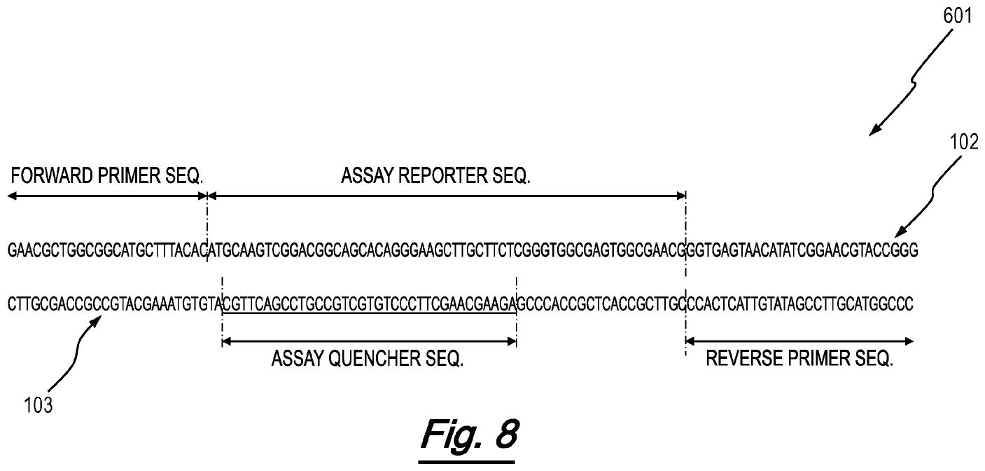

FIG. 8 shows the sequence of a double stranded DNA molecule synthesised in accordance with the procedure of Example 1;

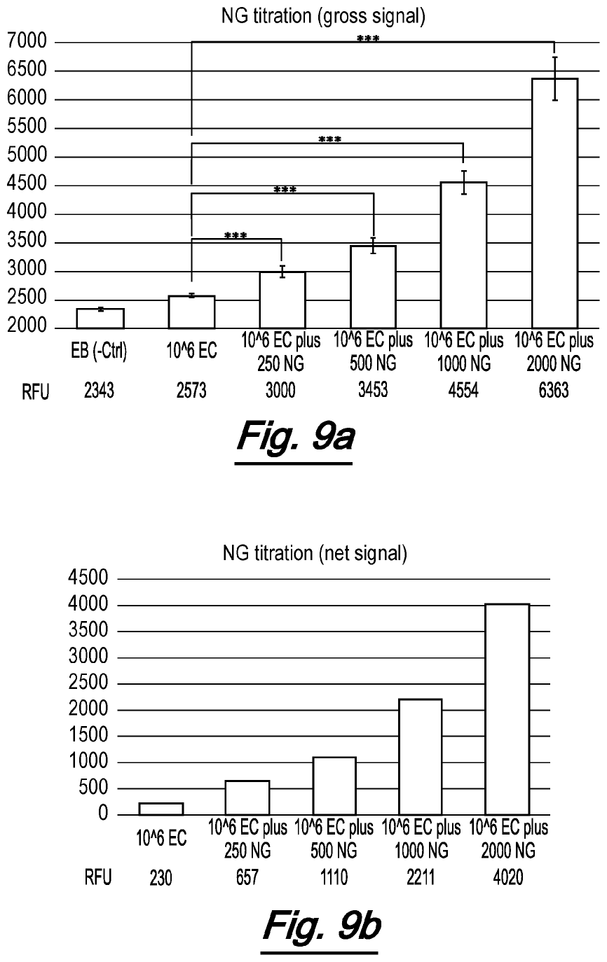

FIG. 9 illustrates the results of Example 1;

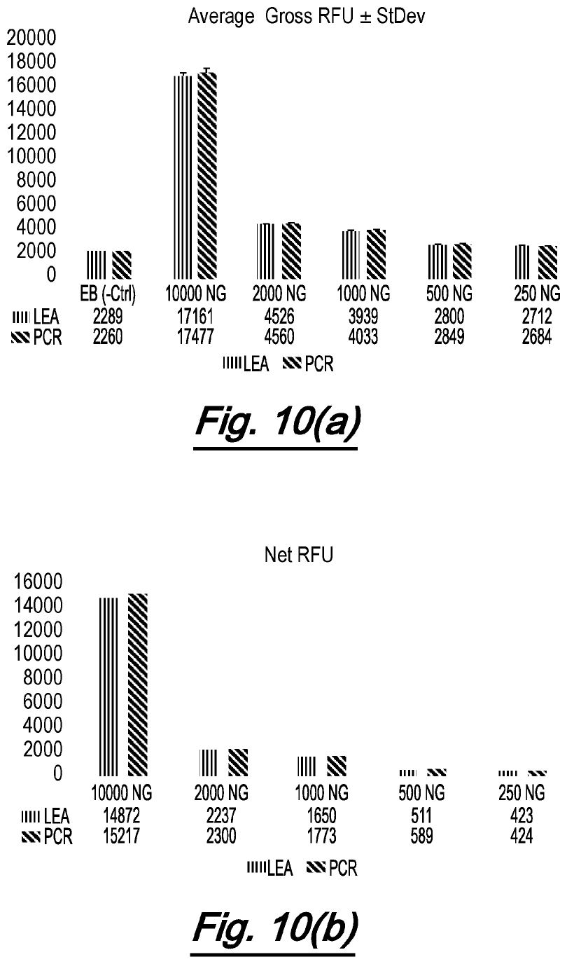

FIG. 10 illustrates the results of Example 2; and

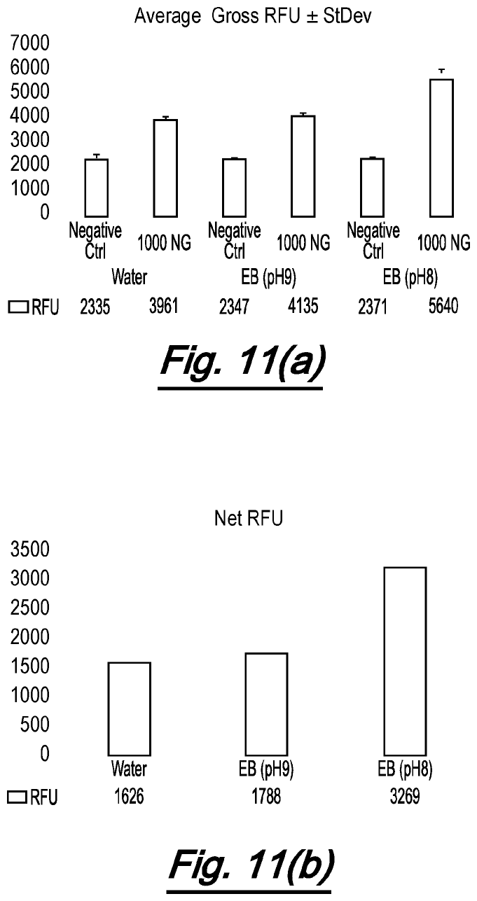

FIG. 11 illustrates the results of Example 3.

Reference is firstly made to FIG. 1 which serves to illustrate the basic concept of the amplification method in accordance with the invention as applied to one embodiment thereof. Represented in FIG. 1 is a double stranded DNA molecule 1 having sense and antisense strands 2 and 3 respectively. For the purposes of the method shown in FIG. 1, the sense strand 2 incorporates the sequence to be amplified. The method of FIG. 1 is for the linear amplification of this sequence.

As shown in FIG. 1, sense strand 2 and antisense strand 3 are hybridised to each other and are of equal length, whereby nucleic acid molecule 1 has blunt ends. For the purposes of explanation, sense strand 2 is considered to have a 5′-end region referenced as 4. This end region 4 extends from the 5′-end of sense strand 2 to a point represented by line 5 (at a position whereof the significance will be appreciated from the subsequent description). The end region may for example be 10 to 15 nucleotides in length. On its 5′ end, sense strand 2 is phosphorylated as shown by the phosphate group (PO₄) clearly depicted in FIG. 1. Intermediate its ends, the 5′-end region 4 of sense strand 2 has a digestion resistant region denoted by the line 6. This digestion resistant region 6 may comprise phosphorothioate (PS) nucleotides (i.e. nucleotides having one of the non-bridging oxygen atoms replaced by a sulfur atom). Although denoted by only a single line, the digestion resistant region 6 will generally comprise several (e.g. three) consecutive modified nucleotides (e.g. phosphorothioate nucleotides). Digestion resistant region 6 may be positioned approximately halfway along end region 4.

Turning now to antisense strand 3, this is a “plain” strand in that it does not have a digestion resistant region. Additionally, the 5′-end of antisense strand 3 is hydroxylated (rather than phosphorylated as in the case of sense strand 2).

Reference is now made to FIG. 2 which shows a primer 10 for use in the method of FIG. 1. Primer 10 is of the same length, and has an identical sequence to, the 5′-end region 4 of sense strand 2. Therefore primer 10 has a phosphate group (PO₄) on its 5′-end and the same digestion resistant region between its ends. For convenience, the digestion resistant region of primer 10 is represented by reference numeral 6 (i.e. the same reference numeral that identifies the digestion resistant region of sense strand 2). Thus, in summary, primer 10 is in effect identical to the 5′-end region of sense strand 2.

To perform the method of FIG. 1, an amplification mixture is prepared which incorporates double stranded nucleic acid molecule 1, primer 10, dNTPs, λ-exonuclease, a strand-displacing polymerase (e.g. BST 3.0) and buffers as appropriate.

In the initial step of the reaction, the λ-exonuclease digests the 5′-end region 4 of sense strand 2 up to (but not beyond) the digestion resistant region 6. This is due to the “ability” of λ-exonuclease to digest one strand of double stranded DNA in the 5′-3′ direction in the following order of preference for the configuration of the ends of the double stranded structure, namely 5′-recessed>blunt<<5′-overhang with a 10× preference for phosphorylated rather than hydroxylated ends. However, the A-exonuclease is not able to effect degradation of sense strand 2 through digestion resistant region 6 thereof. For convenience, the partially digested sense strand is represented by reference numeral 2d. This digestion renders the antisense strand 3 single stranded from its 3′-end over the region to which the now-digested portion of sense strand 2 was previously hybridised. This is clearly depicted in step (ii) of FIG. 1—see the left-hand end of antisense strand 3 which depicts the liberated (i.e. single-stranded) 3′-end region of antisense strand 3 by reference numeral 12.

The liberated 3′-end region of antisense strand 3 forms an overhang 12 which provides a target for hybridisation of primer 10 (see step (iii) of FIG. 1). The strand displacement polymerase extends the hybridised primer 10 to form a new sense strand 2 (using the antisense strand 3 as a template), thereby displacing the partially digested sense strand 2d and regenerating a full length, new sense strand 2 hybridised to antisense strand 3 (see step (iv) of FIG. 1).

It will be appreciated that (since primer 10 has exactly the same sequence (and length) as end region 4 of the sense strand 2 shown in step (i) of FIG. 1) the double stranded molecule 1 depicted as the result of step (iv) of FIG. 1 is identical to that shown in step (i). Therefore the product of step (iv) of FIG. 1 is effectively recycled to step (i) and the method continuously cycles to provide additional displaced strands 2d, whereby there is amplification of a sequence of the sense strand 2.

For the purposes of simplicity, FIG. 1 shows a stepwise mechanism in which each step is “completed” before the next step is commenced. Thus, for example, in going from step (iii) to step (iv), FIG. 1 shows that extension of primer 10 to form new sense strand 2 is complete before the double stranded nucleic acid molecule 1 newly formed in step (iv) undergoes any digestion by the λ-exonuclease. However, we do not preclude the possibility that primer 10 is not fully extended to produce a complete sense strand 2 before digestion (by the λ-exonuclease) of the newly forming strand 2 begins.

It is also possible that since the target molecule is no longer fully double stranded the recess 5′-end strand will dissociate from the antisense strand which will liberate an antisense sequence of greater length that the originally λ-exonuclease/liberated sequence. Although this cannot be proven, it can be presumed considering that the enzyme target is not fully double stranded and the reaction of the method of the invention may take place at relatively elevated temperatures (e.g. 45 to 55° C.) that in theory could induce further dissociation.

Reference is now made to FIG. 3 which illustrates a second embodiment of amplification method in accordance with the invention. The method of FIG. 3 results in exponential amplification. For convenience, FIG. 3 is divided into FIGS. 3(a), 3(b) and 3(c) to facilitate explanation of the invention. Referring firstly to FIG. 3(a), there is shown a double stranded nucleic acid molecule 101 comprised of hybridised sense and antisense strands 102 and 103 respectively. As in the case of double stranded nucleic acid molecule 1 described above with reference to FIG. 1, the sense and antisense strands of nucleic acid molecule 101 are of the same length whereby molecule 101 has blunt ends.

Sense strand 102 is similar to sense strand 2 (of nucleic acid molecule 1) in that it has a 5′-end region 104 that extends from a phosphorylated 5′-end of sense strand 102 to a point depicted by line 105. Intermediate its ends (and approximately halfway therealong) the 5′-end region 104 has a digestion resistant region 106 formed of modified oligonucleotides, e.g. phosphorothioate nucleotides.

Nucleic acid molecule 101 is distinguished from nucleic acid molecule 1 in that (as depicted in FIG. 3a) antisense strand 103 has a 5′-end region 107 extending from the 5′-end of strand 103 (at which there is a phosphate group (PO₄)) to a position designated by line 108. Intermediate its ends, the 5′-end region 107 (of antisense strand 103) has a digestion resistant region depicted by line 109 This digestion resistant region may (as described for the other digestion resistant region) comprise modified oligonucleotides, e.g. phosphorothioate nucleotides.

The reaction of FIG. 3 is effected using primers 110 and 111 as depicted in FIG. 4. Primer 110 has a sequence corresponding to that of the 5′-end region 104 of the sense strand 102 whereas primer 111 has a sequence corresponding to that of the 5′-end region 107 of antisense strand 103. As such, both primers 110 and 111 have (intermediate their ends) digestion resistant regions which are identified (in FIG. 4) by reference numerals 106 and 109 respectively.

In order to effect the reaction of FIG. 3, an amplification mixture is prepared which includes the double stranded nucleic acid molecule 101, the primers 110 and 111, A-exonuclease, a strand displacing polymerase, dNTPs, and buffers as appropriate. The reaction proceeds with the λ-exonuclease partially digesting the strands 102 and 103 from the respective 5′ ends thereof up to (but not beyond) the digestion resistant regions 106 and 109 to produce partially digested strands referenced as 102d and 103d. This action results in a double stranded molecule in which the liberated 3′ ends of the sense and antisense strands form overhangs 113 and 112 respectively.

In the next step of the method, primer 110 hybridises to overhang 112 and primer 111 hybridises to overhang 113. Extension of the hybridised primers 110 and 111 by the strand digesting polymerase leads to the production of the two double stranded molecules referenced as 114 and 115. The double stranded product 114 comprises the residue 102d of sense strand 102 hybridised to a new strand 116 generated by extension of primer 111. The product of Box 6 comprises the residue 103d of antisense strand 103 hybridised to a new strand 117 generated from primer 110.

Further processing of double stranded nucleic acid molecule 114 is shown in FIG. 3b and further processing of double stranded molecule 117 is shown in FIG. 3c.

Referring firstly to FIG. 3b, strand 116 undergoes digestion by the λ-exonuclease from its 5′-end to yield a product in which the residue of strand 116 is depicted as 116d and the 3′-end of strand 102d has formed an overhang 118 to which primer 111 is able to hybridise.

Primer 111 is now extended with concomitant displacement of strand 116d. There are two products of this stage. One is the double stranded molecule 114 (produced by extension of primer 111 using strand 102d as a template). This double stranded molecule is now effectively recycled in the process of FIG. 3b, as depicted by arrow 119. The other product of the reaction is the displaced strand 116d which now hybridises to primer 110. Subsequently primer 110 is extended using strand 116d as a template. This extension is from left to right as seen in FIG. 3b. Also, strand 116d is extended (going from right to left in FIG. 3b) using primer 110 as a template. The resulting product is the double strand molecule depicted as 120 which is shown as being comprised of hybridised strands 121 (formed by extension of primer 110) and 122 (formed by extension of strand 116d).

Strand 121 of double stranded molecule 120 then undergoes digestion (by the action of λ-exonuclease) from its 5′-end up to (but not beyond) the digestion resistant region 106 to provide strand 121d and expose the 3′-end of strand 122 as an overhang 123. As depicted in FIG. 3b, primer 110 is able to hybridise to overhang 123 and be extended using strand 122 as a template, with displacement of strand 121d. The products of this step are, firstly, double stranded molecule 120 and the displaced strand 121d. The former (i.e. double stranded molecule 120) is effectively recycled as depicted by arrow 124 and the latter (i.e. strand 121d) is shown as being associated with arrow 125 which (as described below) leads into part of the scheme shown in FIG. 3c. Also shown in FIG. 3b is arrow 126 which is intended to depict strand 116d (also created in the scheme shown in FIG. 3c—see below) being introduced into the reaction scheme of FIG. 3b. Further description of this aspect of the amplification process will be given below.

Reference is now made to FIG. 3c which, in effect, describes the processing of double stranded molecule 115 (see FIG. 3a) in a manner to the processing of double stranded molecule 114 described fully above in relation to the reaction scheme of FIG. 3b.

Thus, strand 117 undergoes digestion by the λ-exonuclease from its 5′-end to yield a partially digested strand which is identical to strand 121d described above in relation to FIG. 3b. The resulting double stranded product comprises the strand 121d hybridised to strand 103d, with the latter having an overhang 130 to which primer 110 is able to hybridise.

Primer 110 is now extended with concomitant displacement of strand 121d. There are two products of this stage. One is the double stranded molecule 115 (produced by extension of primer 110 using strand 103d as a template). This double stranded molecule is now effectively recycled in the process of FIG. 3c, as depicted by arrow 131. The other product of the reaction is the displaced strand 121d which now hybridises to primer 111. Subsequently primer 111 is extended using strand 121d as a template. This extension is from right to left as seen in FIG. 3c. Also, strand 121d is extended (going from left to right in FIG. 3c) using primer 111 as a template. The resulting product is the double strand molecule depicted as 132 which is shown as being comprised of hybridised strands 133 (formed by extension of primer 111) and 134 (formed by extension of strand 121d).

Strand 133 of double stranded molecule 132 then undergoes digestion (by the action of λ-exonuclease) from its 5′-end up to (but not beyond) the digestion resistant region 109 to provide a partially digested strand which is identical to strand 116d produced in FIG. 3b (see above) and expose the 3′-end of strand 134 as an overhang 135. As depicted in FIG. 3c, primer 111 is able to hybridise to overhang 135 and be extended using strand 134 as a template, with displacement of strand 116d. The products of this step are, firstly, double stranded molecule 132 and the displaced strand 116d. The former (i.e. double stranded molecule 132) is effectively recycled as depicted by arrow 136 and the latter (i.e. strand 116d) is shown as being associated with arrow 126 which (as described below) leads into part of the scheme shown in FIG. 3b. Also shown in FIG. 3c is arrow 125 which is intended to depict strand 121d (created in the scheme shown in FIG. 3b) being introduced into the reaction scheme of FIG. 3c. Further description of this aspect of the amplification process will be given below.

It will be appreciated from the foregoing description that the procedure of FIG. 3b generates single stranded molecule 121d which is also generated in the reaction scheme of FIG. 3c. Thus FIG. 3b shows single stranded molecule 121d being “passed” to the reaction scheme of FIG. 3c as depicted by arrow 125. Similarly, FIG. 3c results in production of single stranded molecule 116d which is also generated in the reaction scheme of FIG. 3b. Thus FIG. 3c shows single stranded molecule 116d being “passed” to the reaction scheme of FIG. 3b, as depicted by arrow 126.

The overall procedure described with reference to FIGS. 3a-c results in an amplified nucleic acid product (as compared to the amount of the original nucleic acid 101 present in the sample.

Reference is now made to FIG. 5 which shows one embodiment of procedure for producing, from an rRNA strand 301, a double stranded nucleic acid molecule 101 of the type shown in FIG. 3(a). As shown in Box 1 of FIG. 5 the illustrated reaction is effected with primers 110 and 111 (see FIG. 3(a) and FIG. 4). Primer 111 is capable of hybridising to the rRNA strand 301 and primer 110 can hybridise to cDNA produced from the rRNA strand 301. Incorporated in the reaction mixture for producing the cDNA is a reverse transcriptase (RT) enzyme that has RNase H activity and also dNTPs and buffers as appropriate. The reaction is effected under thermal cycling conditions.

In the method, primer 111 hybridises to RNA strand 301 and the RT enzyme synthesizes complementary DNA (cDNA) 302 by extension of primer 111 (see Boxes 2 and 3 of FIG. 5). Once the RT enzyme has reached the end of the RNA template strand 301, the RT enzyme reverses direction (as represented by arrow 303) and digests the rRNA template 301 as a result of the enzyme's RNase H activity (see Box 3). In the next step, as shown in Box 4, primer 110 recognises the cDNA sequence and hybridises thereto. Extension of primer 110 by strand displacement polymerase leads to the double stranded construct 304 shown in Box 5 which comprises nucleic acid strand 102 (see FIG. 3(a)) hybridised to the cDNA strand 302. The double stranded construct 304 contains the amplicon sequence of interest plus a 3′ cDNA overhang 305. The two enzymes employed for the purpose of the reaction described in FIG. 3 are now added (i.e. λ-exonuclease and a strand displacing enzyme such as BST 3.0). As will be appreciated from a consideration of the mechanism described with reference to FIG. 3, digestion of the 5′-end of cDNA strand 302 (as far as digestion resistant region 109) allows hybridisation of primer 111 to the liberated 3′-end of strand 102, and extension of primer 111 leads to production of the double stranded nucleic acid molecule 101 with displacement of the cDNA strand 302 (see Box 6). The synthesised nucleic acid construct 101 then undergoes amplification in the manner fully described above in relation to FIG. 3.

It is unlikely that the released cDNA strand will play a further role in the LEA reaction because even though a primer, such as 110, will hybridise and polymerise it will not produce a blunt end on its 5′-end because it will be recessed (long cDNA template 3′-end overhang).

Reference is now made to FIG. 6 which shows one embodiment of procedure for producing, from single stranded DNA 350 (obtained, for example, by denaturation of genomic or plasmid DNA), a double stranded nucleic acid molecule for use in the amplification procedure of the invention. The scheme of FIG. 6 uses three primers 351, 352 and 353 (depicted in dotted lines, long dashed and dotted lines, and medium dashed lines respectively. An example of the long dashed and dotted line is the line immediately underneath the word ‘Primers’ in FIG. 6 and indicated with reference numeral 352. An example of the dotted line is shown the middle of the three lines under the word ‘Primers’ in FIG. 6 and indicated with reference numeral 351. The bottom line under the word ‘Primers’ in FIG. 6 is the medium dashed line and indicated with reference numeral 353). Primers 351 (dotted line) and 353 (medium dashed line) both have digestion resistant regions partway along their respective lengths.

The two reverse primers 351 and 352 (dotted, and long dashed and dotted line) hybridize to strand 350 and polymerise to produce synthetic strands 354 and 355 respectively (dotted, and long dashed and dotted line). The synthetic strand 355 (long dashed and dotted line) displaces synthetic strand 354 (dotted line), providing in this way a template for primer 353 (medium dashed line) to be extended to produce synthetic strand 356 which thereby produces a double stranded molecule 357. This double stranded molecule 357 will be the target for λ-exonuclease which will digest the nuclease-sensitive part of the synthetic strand 354 (dotted line). This will liberate the sequence on the 3′ end of the template strand 356 (medium dashed line) will allow the primer 351 (dotted line) to hybridise and polymerise creating an amplicon as in the final step of FIG. 5. This amplicon can then enter the amplification process. The same system can be used for the rRNA target as well to avoid using the reverse transcriptase (RT) enzyme. For example the two reverse primers 351 and 352 can be added straight to the RNA and since the BST 3.0 polymerase has reverse transcription action it can then produce two strands with strand 355 (long dashed and dotted line) displacing strand 354 (dotted line). Therefore when strand 354 (dotted line) is displaced it can act as a template for primer 353 (medium dashed line). In this case, the RT enzyme's RNase H activity is no longer necessary to digest the RNA template in order to liberate the cDNA strand.

Reference is now made to FIG. 7 which illustrates one embodiment of method for detecting the presence of double stranded amplicons produced in accordance with the method described above in relation to FIG. 3.

In FIG. 7, the amplicons produced by the method of FIG. 3 are depicted by reference numeral 401 and are shown as being comprised of hybridised strands 402 and 403 (shown in dotted lines). At the end of the amplification procedure, there is added to the product mixture a double stranded nucleic acid reporter construct 404 comprised of nucleic acid sequences 405 (shown in dashed and dotted line) and 406 (shown as a solid line) hybridised to each other. Sequences 405 and 406 are capable of hybridising to sequences 402 and 403 respectively of amplicon 401.

As shown in FIG. 7, the 5′-end of sequence 405 is provided with a Cy5 molecule whereas the 3′-end of sequence 406 is provided with a BHQ2 molecule which quenches fluorescence of the Cy5 reporter. (It will be appreciated that other quenched fluorescent combinations may be used).

As shown in FIG. 7, reporter construct 404 has one blunt end provided at the 5′ and 3′ ends of sequences 405 and 406 respectively and the 3′-end of sequence 405 provides an overhang. By way of example, sequence 405 may have a length of 55 nt whereas sequence 406 may have a length of 35 nt. Advantages of this arrangement are discussed below.

The reporter construct 404 is added to the product of the amplification reaction and the mixture raised to elevated temperature (e.g. boiling point) to denature both the double stranded amplicons 401 and the double stranded reporter constructs 404. The mixture is then allowed to cool (e.g. to room temperature) so that sequences 405 are able to hybridise to sequences 402 whereas sequences 406 can hybridise to sequences 403. It will be appreciated that in the double stranded constructs comprising sequence 405 hybridised to sequence 402 the Cy5 is no longer quenched and is therefore capable of providing a fluorescent signal to confirm the presence of the amplified product. It will be appreciated that other hybridisation products are possible (e.g. sequences 402 and 403 may re-hybridise together, similarity sequences 405 and 406) but it is the hybridisation of sequences 402 and 405 that are important for the purposes of detection.

An advantage of the detection procedure shown in FIG. 7 is that unlike molecular beacons that have a recognition sequence of 20 to 30 nt the recognition sequence provided by strand 405 (in the reporter construct 404) can be as long as synthetically possible. A further advantage of the procedure shown in FIG. 7 over molecular beacons is that the loop region of a molecular beacon does not have a competitor oligo (i.e. sequence 406) as in duplex 404. This also makes the system more accurate. More specifically, reporter sequence 404 is thermodynamically more likely to hybridise to the correct rather than the incorrect analyte sequence. This is because the reporter sequence 405 (with its assumed length of 55 nt) will preferentially hybridised to a sequence of the same length in the analyte strand rather than to the (shorter) strand 405 (assumed to be 35 nt). The energy requirement for the full 55 nt of sequence 405 to be hybridised instead of 35 nt (the part of sequence 405 that hybridises to sequence 406) favours hybridisation of sequence 405 to the analyte sequence 402 rather than the sequence 406. On the other hand, if the target sequence is not present in the amplification mixture then the 35 nt homology between sequences 405 and 406 favours hybridisation of these two sequences instead of a “looser bond” between a non-specific analyte and sequence 406.

The invention will be illustrated by the following non-limiting Examples.

### Example

### General

This Example demonstrates detection of Neisseria gonorrhoeae (NG) using procedures in accordance with the present invention. More particularly, the Example demonstrates production of a double stranded DNA molecule from an rRNA extract of NG cells using a procedure in accordance with FIG. 5, amplification of the DNA molecule using a procedure in accordance with FIG. 3, and detection using a procedure in accordance with FIG. 7. To illustrate specificity and sensitivity, the generation, amplification and detection of the DNA molecule were all carried out against a background of excess rRNA from Escherichia Coli (EC).

The double stranded DNA molecule (produced from rRNA extracted from NG) is shown in FIG. 8 and referenced by numeral 601. For comparison with FIG. 3, the two strands of the DNA molecule 601 are referenced as 102 (SEQ ID NO 5) and 103.

Table 1 shows the oligonucleotide sequences used in this Example. By way of explanation, the “forward primer” sequence corresponds with primer 110 in FIG. 3 and the “reverse primer” corresponds with primer 111 in FIG. 2. The “assay reporter” corresponds with sequence 405 in FIG. 7 and the “assay quencher” corresponds with sequence 406 (in FIG. 7).

TABLE 1
Oligo and amplicon sequences table

| Oligo Name | FW | RV | Reporter | Quencher |
|---|---|---|---|---|
| Purpose | Forward primer | Reverse primer | Assay reporter | Assay quencher |
| | (SEQ ID NO 1) | (SEQ ID NO 2) | (SEQ ID NO 3) | (SEQ ID NO 4) |
| Sequence | PO4-5′- | PO4-5′- | 5′-CY5- | 5′- |
| | GAACGCTGGC | CCCGGTACGT | GCAAGTCGGACG | AGAAGCAAGC |
| | G*G*C*ATGC | TC*C*G*ATA | GCAGCACAGGGA | TTCCCTGTGC |
| | TTTACAC-3′ | TGTTACTCAC | AGCTTGCTTCTC | TGCCGTCCGA |
| | | C-3 | GGGTGGCGAGTG | CTTGC-BHQ2- |
| | | | GCGAACG-3′ | 3′ |
| Modifications | Phosphorylation | Phosphorylation | Cy5 molecule | BHQ2 molecule |
| | on 5′ end, 3 PS | on 5′ end, 3 PS | on 5′ end | on 3′ end |
| | linkers (*) in | linkers (*) in | | |
| | the middle of | the middle of | | |
| | the sequence | the sequence | | |

For the purposes of this Example, rRNA was extracted from 100×10⁶ NG cells (determined by total viable count (TVC)) using the procedure described in UK Patent Appln. No. 1609115.9 to give 200 microLt eluate (Elution Buffer (EB):10 mM Tris-HCl, pH9.0, 0.5 mM EDTA). The same procedure was used to obtain a 200 microLt eluate of 100×10⁶ EC cells (determined by optical density (od) measurements).

For the purposes of this Example, it is assumed that all rRNA from both the NG and EC cells is collected in the eluate (i.e. 100% extraction efficiency).

### Procedure

RNA derived from 1×10⁶ EC cells was mixed with RNA derived from 250, 500, 1000 and 2000 NG cells and the volume was made up to 100 microLt. In detail, 2 microLt of the 200 microLt eluate extracted from 100×10⁶ EC cells will contain RNA derived from 1×10⁶ EC cells (on the basis of the above assumption, i.e. 100% extraction efficiency). 2 microLt aliquots of the EC eluate were mixed separately with 2 microLt aliquots of a 1/4000, 1/2000, 1/1000 and 1/500 dilutions of the 200 microLt eluate of the NG RNA (the dilutions containing RNA from 250, 500, 1000 and 2000 NG cells respectively, extracted from 100×10⁶ of NG cells starting material, again assuming 100% extraction efficiency). The final volume was made up at 100 microLt in H₂O, containing final concentrations of 50 microM for each of the two primers (FW and RV), 0.2 mM dNTPs, 4 mM MgSO₄, 20 mM Tris-HCl, 10 mM (NH₄)₂SO₄, 150 mM KCl, 0.1% Tween 20, pH8.8 at 25° C. plus 0.3 microLt (4.5 U) of Warm Start Reverse Transcriptase (New England Biolabs, M0380).

Two negative control conditions were included one with 2 microLt of EB and one condition with only 2 microLt of EC RNA (1×10⁶ EC background control). Each condition was in triplicate.

The samples were left at 48° C. for 10 mins after which 5 microLt of an enzyme mixture: [1 microLt (8 U) BST 3.0 DNA polymerase (New England Biolabs, M0374) plus 0.3 microLt (1.5 U) λ-exonuclease (New England Biolabs, M0262) plus 3.7 microLt of H₂O] was added to each sample and the final mix was left again to incubate at 48° C. for 10 mins.

After the end of this incubation a 10 microLt Assay mix: [Reporter oligo (1 microLt, 10 pmol) plus Quencher oligo (1.4 microLt, 14 pmol) made up at 10 microLt in H₂O containing 15.2 mM Tris-HCl, 7.6 mM (NH₄)₂SO₄, 114 mM KCl, 0.076% Tween 20, pH8.8 at 25° C.] was added to each replicate. The final mix was incubated at 95° C. for 5 mins, then left at room temperature for 2 mins, transferred to black polycarbonate wells and relative fluorescence units (RFU) measurements were taken on a fluorescent plate reader 3× times for each sample.

Values for the 3 reads were averaged for each replicate and these read averages were used for statistical analysis. This experiment was conducted independently 9 times. A one way analysis of variance was conducted using one way ANOVA with post-hoc analysis of variance using an unpaired Student's t-test. NG specific signal vs the non-specific 10⁶ EC signal is significant for all NG titration points (***p≤0.001). Data points are the means of 9 separate experiments conducted in triplicate ±SEM (FIG. 9.a). FIG. 9.b shows all means after subtracting the EB background fluorescence control.

FIG. 9a shows the assay result 30 minutes after purified RNA enters the process. Relative fluorescence unit (RFU) data points of means of 9 independent experiments conducted in triplicate ±SEM. Samples were EB (negative background fluorescence control), RNA from 1 million Escherichia coli (EC) non-specific bacteria and 1 million EC spiked with RNA from 250, 500, 1000 and 2000 Neisseria gonorrhoea (NG) specific bacteria. A one way analysis of variance was conducted using one way ANOVA with post-hoc analysis of variance using an unpaired Student's t-test. NG specific signal vs the EC non-specific signal is significant for all NG titration points (***p≤0.001).

FIG. 9b shows the net signal of means after subtraction of the EB negative background control mean from all other sample means in RFU.

### Example 2

This Example provides a comparison of the amplification method of the present invention with PCR.

A 200 microLt eluate comprising rRNA extracted from 100×10⁶ NG cells was prepared as described in Example 1.

(10 microLt of the extract corresponding to 5 million NG cells (assuming 100% extraction efficiency) was treated with 2 U of DNase I (New England Biolabs, #M0303) in 1× DNase I buffer (10 mM Tris-HCl, 2.5 mM MgCl₂, 0.5 mM CaCl₂, pH 7.6 @ 25° C.) for 30 minutes at 37° C. and the enzyme was denatured at 75° C. for 10 minutes. This was to ensure that no trace of DNA was present in the RNA extract that could affect the PCR result (PCR could amplify the 16S gene as well as cDNA made from 16S rRNA)

Again assuming 100% extraction efficiency, aliquots of the DNase I-treated RNA extract were diluted with water to produce samples representing 1000000, 200000, 100000, 50000 and 25000 NG cells. These samples were then made up to a final volume of 100 microLt in H₂O, containing final concentrations of 50 microM for each of the two primers (FW and RV—see Table 1), 0.2 mM dNTPs, 4 mM MgSO₄, 20 mM Tris-HCl, 10 mM (NH₄)₂SO₄, 150 mM KCl, 0.1% Tween 20, pH8.8 at 25° C. plus 0.3 microLt (4.5 U) of Warm Start Reverse Transcriptase (New England Biolabs, M0380). A negative background fluorescent control sample was prepared in a similar manner from 1 microLt of the elution buffer (EB) used for the RNA extraction procedure.

1 microLt aliquots of each cDNA-containing samples and also the EB were used, as described below, for the purposes of (a) amplification reactions in accordance with the invention, and (b) amplification by PCR.

For the amplification procedure in accordance with the invention, the 1 microLt aliquots were made-up to 100 microLt final volume in H₂O containing: final concentrations of 1.2 microM for each of the two primers (FW and RV), 0.2 mM dNTPs, 4 mM MgSO₄, 20 mM Tris-HCl, 10 mM (NH₄)₂SO₄, 150 mM KCl, 0.1% Tween 20, pH8.8 at 25° C., 8 U BST 3.0 DNA polymerase plus 1.5 U λ-exonuclease. The thus prepared samples therefore represented 10000, 20000, 1000, 500 and 250 NG cells. All samples were prepared (and tested) in duplicate. The amplification reaction was effected at 48° C. for 10 minutes and the product mixture was left on ice until assaying.

For the PCR reaction, the 1 microLt cDNA-containing samples (or the EB control) were diluted to 100 microLt final volume in H₂O containing: 10 mM Tris-HCl, 50 mM KCl, 1.5 mM, MgCl₂, pH 8.3 @ 25° C., 2.5 U Taq Polymerase (New England Biolabs, #0273), 1.2 microM for each of the two primers (FW and RV), and 0.2 mM dNTPs. The PCR amplification protocol was effected using 13 cycles each consisting of denaturation at 95° C., annealing at 57° C. and polymerization at 68° C., with 30 seconds for each temperature (recorded total time of 33 minutes). The product mixture was left on ice until assaying.

The product mixtures obtained (a) using the amplification method of the invention, and (b) amplification by PCR were assayed in the same manner described above in Example 1. The results are shown in FIGS. 10(a) and (b).

FIG. 10(a) shows the Average Gross Relative Fluorescence Units (RFU) ±Standard Deviation (StDev) various sample amplified (and EB control) using both (a) the method of the present invention and (b) amplification by PCR. The data points are the average of the assay results (on duplicate samples) using the two amplification procedures. FIG. 10(b) shows Net RFU values calculated by subtraction of the EB negative control average from all sample averages.

The results in FIGS. 10(a) and 10(b) show that the amplification method of the present invention yielded very similar results to the PCR amplification method. Assuming 100% PCR efficiency, the amplification ratio for 13 cycles is 1 to 8,192 (2¹³). Therefore, the method of the present invention yielded an amplification ratio of about 10⁴ for 10 minutes of isothermal amplification, as compared to a similar result obtained after 33 minutes PCR reaction (which required temperature cycling).

### Example 3

This Example demonstrates the effect of buffer on the amplification yield.

Using the procedure described in Example 1, an RNA extract was prepared from a pellet of extract (starting material a pellet of 100×10⁶ Neisseria gonorrhoeae (NG) lab grown cells eluted using the same elution buffer (EB) as used in Example 1 (pH9).

2 microLt aliquots eluate dilutions dilution in EB corresponding to a total of 1000 NG cells or 2 microLt of EB were added to 81.3 microLt of either H₂O or EB or 10 mM Tris-HCl pH8, 0.5 mM EDTA (“EB pH8”) in duplicate (the rest of the volume to make up to 100 microLt was H₂O with reverse transcription enzyme, and LEA constituents including Tris-HCl in a concentrated format that when diluted to a final volume of 100 microLt was at 20 mM providing a pH 8.8 value.

(Therefore, with the addition of 81.3 microLt of EB or “EB pH8” an extra 8.13 mM of Tris-HCl was added to the reaction making a final concentration of Tris-HCl at 28.13 mM). Reverse transcription, amplification and assaying were carried as in Example 1 (using 4.5 U of the Warm Start Reverse Transcriptase, 8 U of the BST 3.0 DNA polymerase, and 1.5 U of the λ-exonuclease).

The results are shown in FIGS. 11(a) and (b).

In FIG. 11(a), data points are average of assay results for the amp/icons in duplicate with StDev. Templates were negative control (no RNA, 2 microLt of EB pH9) and RNA derived from 1000 Neisseria gonorrhoea (NG) lab grown cells mixed in Water, EB (pH9) or EB (pH8). RFU stands for Relative Fluorescence Units.

In FIG. 11(b), data points are net RFU values calculated by the subtraction of the EB negative control average from all sample averages, revealing EB (pH8) provides optimal LEA yield net RFU values for the net RFU values for the H₂O, EB and 10 mM Tris-HCl pH8, 0.5 mM EDTA mix were 1626, 1788 and 3269 respectively, thus demonstrating the advantage of using EB pH8 as the elution buffer in the RNA extraction protocol.

1. A method of amplifying a nucleic acid sequence comprising:
(a) providing an amplification mixture which comprises:
(i) an exonuclease capable of effecting digestion of a strand of a double stranded nucleic acid molecule with the digestion being from the 5′-end of the strand towards the 3′-end,
(ii) a strand displacing polymerase,
(iii) a double stranded nucleic acid molecule comprising first and second nucleic acid strands hybridised to each other, said first strand incorporating a first nucleic acid sequence to be amplified and having a first 5′-end region which remote from its 5′-end has a nucleotide sequence resistant to digestion (under the conditions of the method) by the exonuclease defined as (i),
(iv) a first nucleic acid primer having the same nucleotide sequence as said first end region of the first nucleic acid, and incorporating the same digestion resistant region, and
(v) nucleotides as appropriate to provide for amplification of the first nucleic acid sequence to be amplified;
and
(b) effecting the amplification reaction under conditions permitting digestion, exonuclease digestion and strand displacement polymerisation thereby producing a product mixture comprising an amplified amount of said first nucleic acid sequence.

2. A method as claimed in claim 1 wherein the 5′-end of the first strand and the 3′-end of the second strand together provide a blunt-end for the double stranded nucleic acid molecule.

3. A method as claimed in claim 2 wherein the 5′-end of the first strand has a 5′-phosphate group and the exonuclease is one which, in the amplification mixture preferentially digests a strand of a double stranded nucleic acid molecule that has a phosphate (PO₄) group at is 5′-end with the digestion being from that end of the strand towards the 3′-end thereof.

4. A method as claimed in claim 3 wherein the exonuclease is λ-exonuclease.

5. A method as claimed in any one of claims 1 to 4 wherein the digestion resistant region of the first end region and of the first primer each comprise at least one phosphorothioate nucleotide.

6. A method as claimed in claim 5 wherein the digestion resistant region of the first end region and of the first primer each comprise a consecutive sequence of a plurality of phosphorothioate nucleotides.

7. A method as claimed in any one of claims 1 to 6 wherein the digestion resistant region of the first end region is intermediate the ends thereof and correspondingly the digestion resistant region of the first primer is intermediate its ends.

8. A method as claimed in claim 1 wherein the second nucleic acid strand has a second 5′-end region which remote from its 5′-end has a nucleotide sequence resistant to digestion (under the conditions of the method) by the exonuclease defined as (i), and the amplification mixture incorporates a second primer identical with said second end region of the second nucleic acid strand.

9. A method as claimed in claim 8 wherein the double stranded nucleic acid molecule has blunt ends.

10. A method as claimed in claim 9 wherein the 5′-ends of each of the first and second strands have a 5′-phosphate group and the exonuclease is one which, in the amplification mixture, preferentially digests a strand of a double stranded nucleic acid molecule that has a phosphate (PO₄) group at is 5′-end with the digestion being from that end of the strand towards the 3′-end thereof.

11. A method as claimed in claim 10 wherein the exonuclease is λ-exonuclease.

12. A method as claimed in any one of claims 8 to 11 wherein the digestion resistant regions of the first end region, of the first primer, of the second end region, and of the second primer each comprise at least one phosphorothioate nucleotide.

13. A method as claimed in claim 12 wherein the digestion resistant regions of the first end region, of the first primer, of the second end region, and of the second primer each comprise a consecutive sequence of a plurality of phosphorothioate nucleotides.

14. A method as claimed in any one of claims 8 to 13 wherein the digestion resistant regions of the first end region and of the second end region are intermediate the ends thereof and correspondingly the digestion resistant regions of the first primer and of the second primer are intermediate their ends.

15. A method as claimed in any one of claims 1 to 14 additionally comprising detecting an amplified sequence.

16. A method as claimed in claim 15 wherein detection is effected by the steps of:
(i) providing in the product mixture a nucleic acid reporter combination which comprises (a) a reporter strand having a fluorescent reporter moiety bound thereto, the reporter strand being capable of hybridising to the amplified nucleic acid sequence to be detected, and (b) a quencher strand capable of hybridising to the reporter strand and having a quencher moiety which quenches the fluorescence of the fluorescent reporter moiety,
(ii) subjecting the product mixture to denaturation and then rehybridisation conditions, and
(iii) detecting for the presence of the fluorescent reporter moiety.

17. A method as claimed in claim 16 when directly or indirectly dependent from any one of claims 8 to 15 for the amplification of a nucleic acid sequence in Neisseria gonorrhoeae wherein the double stranded nucleic acid molecule has the base sequences shown in FIG. 8,
the first primer has the base sequence:
5′-GAACGCTGGCGGCATGCTTTACAC-3′,
the second primer has the base sequence:
5′-CCCGGTACGTTCCGATATGTTACTCACC-3′,
the reporter strand has the base sequence:
5′CY5GCAAGTCGGACGGCAGCACAGGGAAGCTTGCTTCTCGGGTGG
CGAGTGGCGAACG-3′,
and
the quencher strand has the base sequence:
5′-AGAAGCAAGCTTCCCTGTGCTGCCGTCCGACTTGC-3′.

18. A method of determining the presence or otherwise of target nucleic acid sequence in a biological sample, the method comprising the steps of:
(a) processing the biological sample to produce therefrom a derivative sample and under conditions such that, if the target nucleic acid is present in the biological sample, there is generated in the derivative sample a double stranded nucleic acid molecule comprising first and second nucleic acid strands, hybridised to each other, said first strand incorporating a first nucleic acid sequence confirmatory of the presence of target nucleic acid sequence in the biological sample and having a first 5′-end region which remote from its 5′-end has a nucleotide sequence resistant to digestion;
(b) preparing an amplification composition which comprises:
(i) an exonuclease capable of effecting digestion of a strand of a double stranded nucleic acid molecule with the digestion being from the 5′-end of the strand towards the 3′-end,
(ii) a strand displacing polymerase,
(iii) the derivative sample
(iv) a first nucleic acid primer having the same nucleotide sequence as said first end region of the first nucleic acid if present in the derivative sample, and incorporating the same digestion resistant region, and
(v) nucleotides as appropriate to provide for amplification of the first nucleic acid sequence to be amplified; and
(c) analysing for the presence of the first nucleic acid in the product mixture.

19. A method as claimed in claim 18 wherein the 5′-end of the first strand and the 3′-end of the second strand together provide a blunt-end for the double stranded nucleic acid molecule.

20. A method as claimed in claim 19 wherein the 5′-end of the first strand has a 5′-phosphate group and the exonuclease is one which, in the amplification mixture preferentially digests a strand of a double stranded nucleic acid molecule that has a phosphate (PO₄) group at is 5′-end with the digestion being from that end of the strand towards the 3′-end thereof.

21. A method as claimed in claim 20 wherein the exonuclease is λ-exonuclease.

22. A method as claimed in any one of claims 18 to 21 wherein the digestion resistant region of the first end region and of the first primer each comprise at least one phosphorothioate nucleotide.

23. A method as claimed in claim 22 wherein the digestion resistant region of the first end region and of the first primer each comprise a consecutive sequence of a plurality of phosphorothioate nucleotides.

24. A method as claimed in any one of claims 18 to 23 wherein the digestion resistant region of the first end region is intermediate the ends thereof and correspondingly the digestion resistant region of the first primer is intermediate its ends.

25. A method as claimed in claim 18 wherein the second nucleic acid strand has a second 5′-end region which remote from its 5′-end has a nucleotide sequence resistant to digestion (under the conditions of the method) by the exonuclease defined as (i), and the amplification mixture incorporates a second primer identical with said second end region of the second nucleic acid strand.

26. A method as claimed in claim 25 wherein the double stranded nucleic acid molecule has blunt ends.

27. A method as claimed in claim 26 wherein the 5′-ends of each of the first and second strands have a 5′-phosphate group and the exonuclease is one which, in the amplification mixture, preferentially digests a strand of a double stranded nucleic acid molecule that has a phosphate (PO₄) group at is 5′-end with the digestion being from that end of the strand towards the 3′-end thereof.

28. A method as claimed in claim 27 wherein the exonuclease is λ-exonuclease.

29. A method as claimed in any one of claims 25 to 28 wherein the digestion resistant regions of the first end region, of the first primer, of the second end region, and of the second primer each comprise at least one phosphorothioate nucleotide.

30. A method as claimed in claim 29 wherein the digestion resistant regions of the first end region, of the first primer, of the second end region, and of the second primer each comprise a consecutive sequence of a plurality of phosphorothioate nucleotides.

31. A method as claimed in any one of claims 25 to 30 wherein the digestion resistant regions of the first end region and of the second end region are intermediate the ends thereof and correspondingly the digestion resistant regions of the first primer and of the second primer are intermediate their ends.

32. A method as claimed in any one of claims 18 to 31 wherein detection is effected by the steps of:
(i) providing in the product mixture a nucleic acid reporter construct which comprises (a) a reporter strand having a fluorescent reporter moiety bound thereto, the reporter strand being capable of hybridising to the amplified nucleic acid sequence to be detected, and (b) a quencher strand hybridised to the reporter strand and having a quencher moiety which quenches the fluorescence of the fluorescent reporter moiety,
(ii) subjecting the product mixture to denaturation and then rehybridisation conditions, and
(iii) detecting for the presence of the fluorescent reporter moiety.

33. A method as claimed in claim 32 when directly or indirectly dependent from any one of claims 25 to 31 for the detection of a nucleic acid sequence in Neisseria gonorrhoeae wherein the double stranded nucleic acid molecule, if present, has the base sequences shown in FIG. 8,
the first primer has the base sequence:
5′-GAACGCTGGCGGCATGCTTTACAC-3′,
the second primer has the base sequence:
5′-CCCGGTACGTTCCGATATGTTACTCACC-3′,
the reporter strand has the base sequence:
5′CY5GCAAGTCGGACGGCAGCACAGGGAAGCTTGCTTCTCGGGTGG
CGAGTGGCGAACG-3′,
and
the quencher strand has the base sequence:
5′-AGAAGCAAGCTTCCCTGTGCTGCCGTCCGACTTGC-3′.

34. A method of detecting a nucleic acid in a sample to be analysed, the method comprising the steps of:
(i) providing in the sample a nucleic acid reporter combination which comprises (a) a reporter strand having a fluorescent reporter moiety bound thereto, the reporter strand being capable of hybridising to the nucleic acid sequence to be detected, and (b) a quencher capable of strand hybridising to the reporter strand and having a quencher moiety which quenches the fluorescence of the fluorescent reporter moiety,
(ii) subjecting the mixture to denaturation and then rehybridisation conditions, and
(iii) detecting for the presence of the fluorescent reporter moiety.


SEQUENCE LISTING (5 sequences)

SEQ ID NO: 1 - DNA, 24 bp, Artificial Sequence
  FW  forward primer
  misc_difference (1)..(1): Phosphorylation on 5′ end
  modified_base (11)..(13): phosphorothioate (PS) nucleotides
gaacgctggcggcatgctttacac

SEQ ID NO: 2 - DNA, 28 bp, Artificial Sequence
  RV reverse primer
  misc_difference (1)..(1): Phosphorylation on 5′ end
  modified_base (12)..(14): phosphorothioate (PS) nucleotides
cccggtacgttccgatatgttactcacc

SEQ ID NO: 3 - DNA, 55 bp, Artificial Sequence
  assay reporter oligonucleotide
  misc_difference (1)..(1): Cy5 molecule on 5′ end
gcaagtcggacggcagcacagggaagcttgcttctcgggtggcgagtggcgaacg

SEQ ID NO: 4 - DNA, 35 bp, Artificial Sequence
  assay quencher oligonucleotide
  misc_difference (35)..(35): BHQ2 molecule on 3′ end
agaagcaagcttccctgtgctgccgtccgacttgc

SEQ ID NO: 5 - DNA, 109 bp, Neisseria gonorrhoeae
gaacgctggcggcatgctttacacatgcaagtcggacggcagcacagggaagcttgcttctcgggtggcgagtggcgaacgggtgagtaacatatcggaacgtaccggg